(19) United States
(12) Patent Application Publication
(10) Pub. No.: US 2008/0154219 A1
(43) Pub. Date: Jun. 26, 2008

(54) Bodily fluid collection bag kit with externally attached cleansing wipe and breath freshener

(76) Inventors: Jeffery S. Longo, Jacksonville, FL (US); Kim L. Longo, Jacksonville, FL (US)

Correspondence Address: Gary R. Molnar, Suite 10A, 138 East 38th Street, New York, NY, 10016-2646, US

(21) Appl. No.: 11/704,606

(22) Filed: Feb. 9, 2007

Related U.S. Application Data
(60) Provisional application No. 60/876,622, filed on Dec. 21, 2006.

(51) Int. Cl. A61M 1/00

(52) U.S. Cl. 604/327

(57) ABSTRACT

A bodily fluid collection kit comprising a disposable bag container and a cleansing towelette and breath freshener removably contained at or attached to an outside surface of the bag, that is useful for the collection of bodily fluid and the cleansing and refreshment of the user.

### FIELD OF THE INVENTION

This invention relates generally to a disposable bag for the collection of bodily fluid, and more particularly to the collection and disposal of emesis.

### BACKGROUND OF THE INVENTION

The present invention provides an economical, efficient, convenient and simple containment, cleansing and freshening means for the collection of bodily fluid, its disposal, and the personal cleansing and freshening of the user.

Many people become afflicted with a temporary malady commonly known as motion sickness. Motion sickness is brought about by unevenness of motion especially that common in airplanes and on ships. Symptoms of motion sickness include nausea which may progress to vomiting. Accordingly, virtually every passenger airplane and ship carries containment devices so that, at the onset of nausea, passengers will have a container in which to regurgitate should the nausea progress to vomiting. Most typical of these containment devices is simple paper or plastic bags that are commonly known as motion sickness bags.

Other environments where there may be concerns of nausea and vomiting are in automobiles, small watercraft, amusement parks, play grounds and other areas where motion sickness may occur.

There also are many other conditions which may cause a person to become nauseous with a need to regurgitate such as high anxiety, nervousness, claustrophobia, pregnancy, and gastrointestinal disorders.

Thus, other environments in which a readily available containment device in which a person may regurgitate may be needed are where a person is infirmed or bed-ridden, doctor offices, dentist offices, hospitals, and hospital equipment (such as MRI & CAT scan units), company clinics, schools, decompression and hyperbaric chambers, elevators, space travel, space travel training facilities and other places where a person finds himself or herself in a confined or remote location and may become nauseous or with a need to regurgitate but without any conventional facilities readily available.

Hospitals often employ metallic “kidney dishes” for the collection of bodily fluids. These dishes are not closeable and are not accompanied with a readily available cleansing means or a freshener.

Various improvements in the design of the basic motion sickness bag and other bodily fluid receptacles have been proposed. For example, U.S. Pat. Nos. 7,041,042, and 7,029,463 are directed to improved container or containment devices. The disclosures, however, are only directed to issues of leakage of fluid or odor, or sound suppression. They do not address the needs of the ill person for comfort, cleansing and refreshing. The act of vomiting inherently creates issues and needs relating to well-being, embarrassment, cleanliness, personal hygiene and mouth and breath refreshment.

Currently, there apparently is no item on the market that provides convenient and simple means for the collection of bodily fluid with consideration for the user's comfort, hygiene, and refreshing; or, most particularly, for providing for personal hygiene and refreshment in connection with the collection and disposal of vomitus.

Thus, there is a need for a convenient and simple means for the collection of bodily fluids (particularly emesis) that provides the user with a readily available cleansing towelette and a breath freshener for the user's hygiene and refreshment.

The present invention addresses this need for a simple and convenient means for collecting bodily fluid and provides a readily available cleansing means and breath freshener to the afflicted in a self-contained bodily fluid collection bag kit for the collection of bodily fluid and for personal hygiene and freshness.

Another object of the invention is to incorporate and make readily available a cleanser and a breath freshener by their convenient attachment to a bodily fluid collection bag.

A further object is to provide a collection bag kit that, before used, is small, compact, and light-weight yet contains personal hygiene articles that are attached and readily available to the user.

Yet another object of the invention is to provide absorption of bodily fluid within a bag container to reduce the risk of spillage or leakage of the bodily fluid from the bag container.

An additional object of the present invention is to provide a bodily fluid collection bag kit that is disposable and environmentally friendly by means of producing the invention with materials that are biodegradable or compostable.

### SUMMARY OF THE INVENTION

The present invention is a bodily fluid collection bag kit comprising a disposable bag container with a mouth opening, and a cleansing towelette and breath freshener removably attached to an outside surface of the bag container.

Attachment of a cleansing towelette and a breath freshener to an outside surface of the bag container provides a means for personal hygiene and cleansing, and a means for freshening, that are readily and conveniently accessible to the user.

Thus, the present invention provides an economical, efficient and sanitary means for the collection of bodily fluid, (particularly emesis), its disposal, and the subsequent cleansing of the person using the invention and their refreshment.

A preferred embodiment of the bodily fluid collection bag kit of the present invention comprises a bag container having a bottom edge opposite a mouth opening, and one or more side walls extending from the mouth opening to the bottom edge. To one or more of the side walls' outside surfaces are removably attached a cleansing towelette and a breath freshener.

In another preferred embodiment of the present invention, the bodily fluid collection bag kit of the present invention comprises a bag container having a bottom wall opposite a mouth opening, and one or more side walls extending from the mouth opening to the bottom wall. To the outside surface of the bottom wall are removably attached a cleansing towelette and a breath freshener.

In a preferred embodiment the bodily fluid collection bag kit has a folded accordion style configuration. In this embodiment the collection bag elongates as the bodily fluid enters to contain the amount of bodily fluid entering. The bodily fluid collection bag kit of this embodiment comprises a disposable, flexible bag container having an interior with a mouth opening, a bottom wall opposite the mouth opening, and one or more side walls extending from the mouth opening to the bottom wall which at least partially define the interior of the bag container. The one or more side walls each have one or more inward pleats overlying the bottom wall. A breath freshener and a cleansing towelette are removably attached to an outside surface of the bottom wall.

In the present invention it is preferred that the bodily fluid collection bag kit contains an absorbent material, preferably a gelling material, inside the bag container, such that when a bodily fluid enters the bag through the mouth opening at least some of the bodily fluid is absorbed thereby reducing the risk of seepage or leakage. When the absorbent material is a gelling material, it turns into a gel solidifying the bodily fluid, or at least making it more viscous. The gelling material is preferably a cellulose material or a hydrophilic material and preferably a polymer.

In a preferred embodiment the bag container is made of a material that is impermeable to the absorbent material (e.g. a gelling material) and the bodily fluids. The bag container material may be coated paper (coated with a wax or plastic film) that is impermeable to bodily fluids. Alternatively, the bag container material is polyethylene or polypropylene. Most preferred is that the bag kit is biodegradable or compostable.

In the present invention, removably attached to, or contained on an outside surface of the bag container is a cleansing towelette to provide a ready means for the user to cleanse as needed. The attached cleansing towelette is preferably pre-moistened with a wet cleanser that is sanitizing or antimicrobial.

Also attached to or contained on an outside surface of the bag container is a breath freshener. The attached freshener is provided to refreshen the user's breath or to make the user's mouth feel clean and refreshed. The freshener may be an edible film, orally dissolving strip, chewing gum, tablet, lozenge, liquid, hard candy, a gel, or any other suitable breath freshener medium. Preferred is an orally dissolving strip as it is believed more suited in packaging the present invention.

The preferred means of removably attaching or containing the cleansing towelette or wipe and the breath freshener to on outside surface of the bag container is to provide a peel-away cover layer over the items on an outside surface of the bag container. The cover layer may be formed by placing the cleansing towelette and breath freshener against an outside surface of the bag container material, laying a sheet of the bag material or another material on top of the cleansing towelette and breath freshener, and then heat or pressure sealing the bag materials together, or by using an adhesive to adhere the cover layer to an outside surface of the bag container.

The bodily fluid collection bag kit preferably has a closure means for sealing the mouth opening of the bag container after the bag container is used. The closure means may be adhesive or mechanical, such as a twist tie or drawstring, or preferably a zipper seal.

The bodily fluid collection bag kit of the present invention may be made of biodegradable or compostable material. Thus, the present invention may be made, used and disposed environmentally responsibly.

Typical uses for the invention include the collection and disposal of emesis.

In addition, the present invention may be utilized in other areas of bodily fluid collection. Such additional uses include, as an example, the collection of urine and excrement. The collection bag kit would be utilized in needed quick accommodating fashion in the event a person has suddenly become ill and needs to urinate or has a sudden attack of diarrhea. The collection bag kit would allow the attention to hygiene by means of the attached wipe.

For a better understanding of the present invention, together with other and further embodiments, reference is made to the following description taken in conjunction with the Figures, the scope of which is set forth in the appended claims.

### BRIEF DESCRIPTION OF THE DRAWINGS

FIG. 1 is a schematic of an embodiment of the invention comprising a bag container having attached to an outside surface a sanitizing wipe and a breath freshener.

FIG. 2 is a sectional view of the embodiment of FIG. 1 taken along line 13-13 of FIG. 1.

FIG. 3 is a schematic sectional view taken along line 33-33 of FIG. 4 of a second embodiment of the invention comprising a bag container in an accordion-style folded configuration having attached to an outside surface of a bottom wall a sanitizing wipe and a breath freshener, illustrated slightly expanded to show construction details.

FIG. 4 is a schematic view of the bottom outside surface of the embodiment of FIG. 3.

Where used in the various figures, the same numerals designate the same or similar parts.

### DETAILED DESCRIPTION OF THE INVENTION

In preparing the preferred embodiments of the present invention, various alternatives may be used to facilitate the objects of the invention. These embodiments are presented to aid in an understanding of the invention and are not intended to, and should not be construed to limit the invention in any way. All alternatives, modifications and equivalents that may become obvious to those of ordinary skill upon a reading of the present disclosure are included within the spirit and scope of the present invention.

Further, this disclosure is not a primer on processes or materials for making bag containers, towelettes, packets, breath freshener, or collection bag kits; basic concepts known to those skilled in the art have not been set forth in detail. Readers are referred to appropriate and available texts for further details on these subjects.

Briefly stated, the bodily fluid collection bag kit of the present invention comprises a disposable bag container along with a cleansing towelette and breath freshener that are removably attached to, or contained at, an outside surface of the bag container.

This invention allows the expelling of bodily fluids into a bag for containment, and attends to cleansing, personal hygiene attention and breath refreshment with the availability of an attached cleansing wipe and an attached breath freshener.

Particularly preferred embodiments of the invention are illustrated by FIGS. 1 and 2 and FIGS. 3 and 4.

The bodily fluid collection kit of the embodiment illustrated by FIG. 1 includes a generally rectangular flexible bag container 1 which has removably attached to an outside surface 2 a generally rectangular removable, peel-away cover ply of transparent plastic 3 with its outer edges 4a, 4b, 4c and 4d pressured sealed to the outside surface 2 of the bag container. The cover ply 3 also has an edge 5 for the user to grasp to peel away the cover ply. Contained between the outer surface 2 and the cover ply 3 are a sanitizing wipe 6 with an antimicrobial solution within a packet 7 and a breath freshener strip 8 within a packet 9. The bag container 1 has a mouth opening 10 that is sealed with a re-sealable adhesive 11. Opposite the mouth opening is the bottom edge 12 of the bag container.

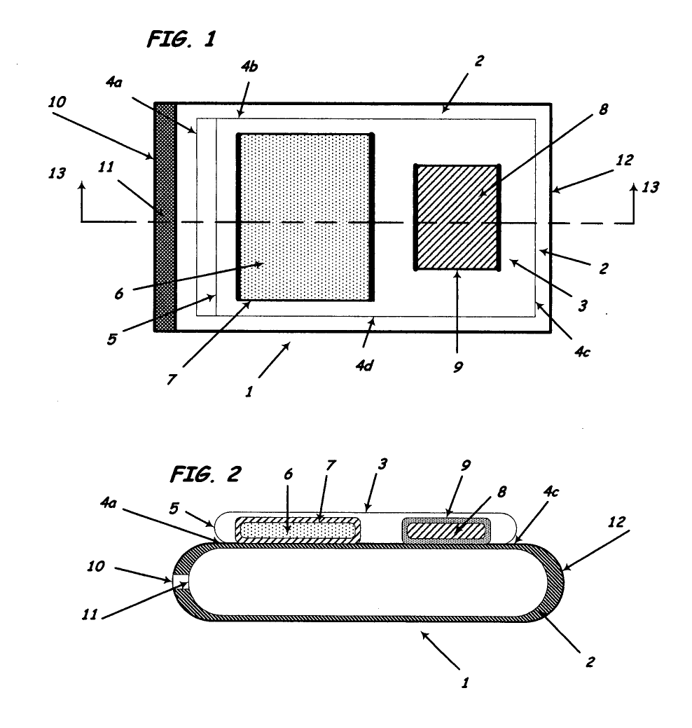

FIG. 2 further shows the attachment to the outside surface 2 of bag container 1 of the outer edges 4a and 4c of the cover ply 3, and the containment of the sanitizing wipe 6 in packet 7, and the breath freshener 8 within packet 9.

The bag container of the bodily fluid collection kit of the present invention illustrated by FIGS. 1 and 2 may have a lateral fold or folds and be folded laterally. The bag container of the bodily fluid collection kit also may have longitudinal fold or folds and be folded longitudinally from the bottom edge to the mouth. A folded bag container provides for a compact collection kit that may be stored in places of limited space such as passenger seat pockets, access panels and similar type compartments that contain slots, pockets or other small areas which permit for stowage of only small compact items such as magazines, keys, cell phones, and other related compact size items.

In use, the bodily fluid collection kit is removed from its storage location upon the initial onset of nausea and, if in a folded configuration, opened from its folded, compact configuration. The user then opens the mouth of the container bag and holding the container bag to their mouth, vomitus may be expelled into the container bag. After regurgitating into the container bag, the user then may peel away the cover ply and conveniently access the towelette for cleansing and the breath freshener as needed or desired. Once the container bag has been used, the used towelette and breath freshener and their respective packets can be placed in the container bag, the container bag is sealed or resealed, and the container bag be properly disposed at an appropriate time.

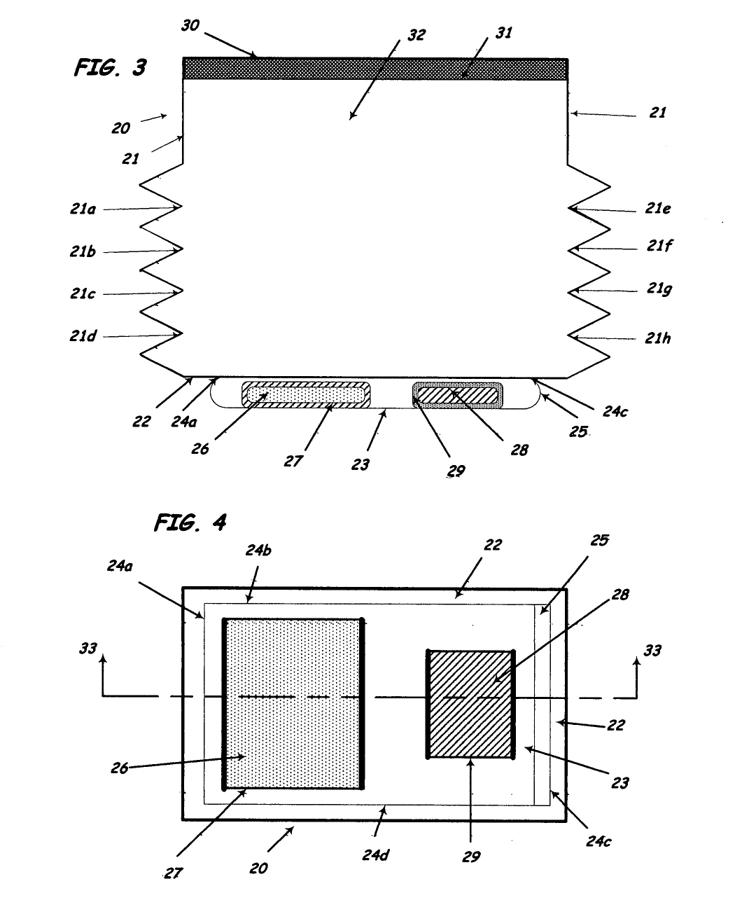

Another particularly preferred embodiment of the invention is illustrated by FIGS. 3 and 4. In the illustrated embodiment, the bodily fluid collection kit includes a generally rectangular flexible bag container 20 which has side wall 21 and a bottom wall 22. The side wall 21 and the bottom wall 22 at least partially define the interior 32 of the bag container. Opposite the bottom wall 22, the bag container 20 has a mouth opening 30 that is sealed with a re-sealable adhesive 31. The side wall 21 includes gussets or pleats 21a, 21b, 21c, 21d, 21e, 21f, 21g and 21h that enable the bag container to be folded flat for storage. In its collapsed, flat-folded, pre-use, state, the gusseted side wall 21 is folded inwardly, accordion-style, such that pleats 21a and 21e overlie pleats 21b and 21f which overlie pleats 21c and 21g which in turn overlie pleats 21d and 21h that overlie bottom wall 22. Additional or fewer gussets or pleats may be incorporated. In use, the inflow of bodily fluid causes the pleats 21a, 21b, 21c, 21d, 21e, 21f, 21g and 21h to outwardly unfold and enable the side wall 21 to elongate and expand the interior 32 of the bag container 20 to contain the inflowing bodily fluid. To the outside surface of bottom wall 22 is a removably attached, generally rectangular, peel-away cover ply of transparent plastic 23. Contained between the outer surface of wall 22 and the cover ply 23 is a sanitizing wipe or towelette 26 within a packet 27 and a breath freshener 28 within a packet 29.

FIG. 4 further illustrates the attachment of the cover ply 23 to the outside surface of bottom wall 22. Cover ply 23 is removably attached by its outer edges 24a, 24b, 24c and 24d pressured sealed to the outside surface of the bottom wall 22 of the bag container. As in the embodiment illustrated by FIGS. 1 and 2, the cover ply 23 also has an edge 25 for the user to grasp to peel away the cover ply. Contained between the outer surface of bottom wall 22 and the cover ply 23 are the sanitizing wipe 26 with an antimicrobial solution within a packet 27 and the breath freshener strip 28 within a packet 29.

The embodiment of the present invention illustrated by FIGS. 3 and 4 may have, in addition to the side wall pleats or gussets, a lateral or longitudinal fold or folds and be laterally or longitudinally folded to further compact the collection kit. The bag container of the bodily fluid collection kit also may be folded longitudinally from the bottom wall to the mouth opening to create an inwardly directed pleat or gusset.

The accordion-style folded embodiment illustrated by FIGS. 3 and 4 may be used in the same or similar manner as the embodiment illustrated by FIGS. 1 and 2. The user opens the mouth of the bag container and holding the bag container to their mouth, vomitus may be expelled into the bag container. In this embodiment, inflowing bodily fluid causes gusseted side wall 21 to expand, outwardly unfolding and separating the pleats 21a, 21b, 21c, 21d, 21e, 21f, 21g and 21h, thereby extending the length of the side wall 21 of bag container 20, and increasing the bag container's interior volume.

Preferably, in this embodiment, the bag container expands in relative proportion to the amount of inflowing bodily fluid.

Having illustrated the present invention by describing specific preferred embodiments with reference to the drawings, various alternate embodiments are now described.

The disposal bag container employed in the present invention may be of standard construction, with the exception that a cleansing towelette and breath freshener are removably attached to an outside surface of the bag.

The disposable bag container has a mouth opening. The mouth opening is of sufficient size to substantially cover the intended user's mouth. The mouth opening may be initially sealed or open. Opposite the mouth opening is the bottom edge or side, or bottom wall of the collection bag container.

The bag container of the bodily fluid collection kit may be comprised of a single sheet of material. The sheet may be folded to form a tubular bag stock that defines the side, bottom and mouth opening of the bag container. The bottom of the bag container may be an edge (e.g. having only a linear dimension; such as the construction of a typical plastic “baggie” food storage bag) or a wall (having a width and depth, such as the construction of a typical plastic sandwich bag). The bottom edge or wall is opposite the mouth opening and the side extends from the bottom edge or wall to the mouth opening.

The bag container also may be comprised of a single sheet of material whose side edges and bottom edge are joined to form the bag container either with a bottom edge or a bottom wall (e.g. the construction of a typical paper magazine bag or a paper sandwich bag).

Two opposing side walls, or more sets of opposing side walls, may be provided with their edges joined to form the bag container of the bodily fluid collection kit.

The side wall or walls extending from the bottom edge or wall to the mouth opening at least partially define the interior of the bag container.

A cleansing wipe or towelette and a breath freshener are removably attached, or contained on an outside surface of the bag container. When the bag container has a bottom edge rather than a bottom wall, the cleansing towelette and the breath freshener are attached to, or contained on an outside surface of the side wall or walls as there is no, or substantially no, bottom outside surface to which to attach or contain the items. If a bottom wall is provided, the cleansing towelette and breath freshener may be attached to, or contained on an outside surface of the bottom wall or the side wall or walls.

The side wall or walls of the bag container of the bodily fluid collection bag preferably has one or more lateral inward folding pleats or gussets. Fold lines are provided across the side wall or walls substantially parallel to the bottom edge or wall to enable the formation of a gusset or gussets in the side wall or walls that may be folded inwardly in an accordion-style pleat that overlies the bottom of the bag container. The gusseted side wall enables the wall to collapse and the bag container to be folded flat for compact storage prior to use. When in use, as bodily fluid enters the bag container, the gusset or gussets unfold outwardly and away from the bottom of the bag container expanding the length of the side or side walls, elongating the bag container and increasing its interior volume to contain the inflowing bodily fluid.

The sides of the bag container may be similarly folded or pleated longitudinally from the bottom edge or side to the mouth. That is, in addition, or alternative to lateral gussets or pleats, the side wall or side walls may be provided with one or more longitudinal gussets or pleats running from the bottom edge or wall to the mouth opening. Fold lines are provided substantially perpendicular to the bottom edge or wall along the length of the side wall or walls from the bottom edge or wall to the mouth opening to create one or more inwardly directed pleats or gussets.

The bag container of the bodily fluid collection kit also or alternatively may be folded without creating pleats or gussets, either laterally, longitudinally, or both.

The bag container may also have a folded or pleated bottom side or wall that spreads apart.

The provision of pleats or gussets, either lateral or longitudinal, or folds, either lateral or longitudinal, enables a compact collection kit that may be stored in a limited space yet be easily unfolded or expanded in use to contain bodily fluids.

In a particularly preferred embodiment, the bag container has pleats or gussets that allow it to expand to contain the amount of bodily fluid entering. The embodiment comprises a disposable, flexible bag container comprising a bottom wall, a mouth opening opposite the bottom wall, and two opposing side walls. The bottom wall and the first and second side walls at least partially define the interior space of the bag container. Each of the side walls has a plurality of lateral pleats or gussets that fold accordion-style inwardly to overly the bottom wall of the bag container and collapse the side walls and allow the bag container to fold flat. The pleats and gussets unfold outwardly at least partially as bodily fluid flows into the interior of the bag container, elongating the side walls and thereby increasing the volume of the bag container's interior. A cleansing towelette and a breath freshener are attached to an outside surface of the bottom wall of the bag container. A bodily fluid collection bag kit comprising an expansible bag container having an accordion-style folded construction with a cleansing towelette and breath freshener removably attached to the outside surface of the bottom of the bag is particularly preferred as it has a compact size in its collapsed state, it allows for expansion of the bag containers interior volume with the inflow of bodily fluid, and it provides a conveniently accessible cleansing towelette and breath freshener on an outside bottom surface of the bag container.

The bag container of the bodily fluid collection kit may be folded laterally to form a compact collection kit.

The bag container may be made of any suitable flexible material. The bag container, for instance, may be made of folded heavy paper that may be lined on its interior with a finish to resist leakage or seepage. The finish may be a wax type coating or a plastic film. The bag container material is preferable impermeable to bodily fluids.

More preferably, the material will be a film sheet. It is preferable that the material be moisture proof and odor impermeable. Such materials include polymer blend plastic material, particularly polyethylene or polypropylene.

In preferred embodiments, an absorbent material, preferably a gelling material, is provided inside the bag container. The bag container of these embodiments is made of a material that is impermeable to the absorbent material and the bodily fluids. The bag container material may be coated paper (coated with a wax or plastic film) that is impermeable to the absorbent material and bodily fluids. Alternatively, the bag container material is polyethylene or polypropylene.

For environmental reasons, it is preferred that the bag container be made of material that is biodegradable; more preferably, a material that is compostable. A biodegradable material is any material made of a bio-base (such as corn, wheat, potatoes, etc.) which, when disposed of after use, will be decomposed completely by micro-organisms generally present in the earth into carbon dioxide, water and biomass. Preferably, the biodegradable material meets one or more of the definitions set forth in The European Norm EN13432, Japanese Greenpla Standard and the American Society for Testing and Materials (ASTM International) D6400-99 standards.

A compostable material is a material that will biodegrade in or otherwise become part of, usable compost (e.g., soil-conditioning material) in a safe or timely manner in an appropriate composting program.

Biodegradable and compostable bags and bag material that are suitable in the manufacture of the present invention include those available from Biocorp, Inc., Becker, Minn.; BIOgroupUSA, Jacksonville, Fla.; and BioBag International, Askim, Norway. These materials employ Mater-Bi™ materials which are a family of biodegradable thermoplastic materials manufactured by Novamont SpA, Novara, Italy.

The bag container preferably has a volume that is more than adequate to accommodate the severest of motion sickness bouts. It is preferred the bag container size be approximately, but not limed to, about 25.4 centimeters (ten inches) in length and about 16.5 centimeters (six and one half inches) in width. When the bag container is provided with a bottom wall it is preferred that, when in its open or expanded condition, the depth of the bag container at its bottom be about 16.5 centimeters (six and one half inches).

The bag containers are shown in the Figures as having a generally rectangular shape. It will be evident that it may also be tapered, have a rounded or pointed bottom edge or have some other desired configuration, as long as the containment and disposal functions are adequately maintained.

In embodiments having a bottom wall, the bottom wall may be generally rectangular, circular, oval, or any other desired shape, provided that, if the cleansing wipe or breath freshener are to be attached to or contained at the outside surface of the bottom wall, the bottom wall has an outside surface sized and shaped to meet that requirement.

The bag container has a mouth opening that initially may be sealed completely or be left open. It is preferred that the bag container be initially sealed. If completely sealed, means are provided for the user to readily open the bag container. Any ready open sealing means of any suitable mechanical, heat or pressure, or adhesive type may be employed; preferred is an adhesive seal. An adhesive seal includes seals employing glue, tape, or materials specifically formulated for forming a seal. Such materials may be formulated Methacrylates, Acrylics, Polyurethane, Cyanocrylate and UV Curable adhesive. Such preferred ready open means include a perforation along an adhesive seal that can be grasped and pulled in a side-to-opposite side manner in order to open the mouth.

Other suitable ready open seals include those commonly used on plastic sandwich or storage bags, such as a mechanical method type of a zipper seal (a raised ridge on one inner side that fits inside a groove on an opposite inner side), a slider zipper (that uses the same raised ridge and inside groove, but seals by sliding a tab down the length of the opening), or a twist tie.

To prevent spillage or leakage of material collected or the release of odor from the bag container before its disposal, a closure means for sealing or resealing the bag container once the bodily fluid is inside is preferably provided along an outer surface at or near the mouth opening. If the bag container is initially provided sealed, the closure means may be the same, different or integral with the initial sealing means. Such means include those described above. Other suitable closure means include a pull away tape attached on the outside of the bag container. The tape covering would be pulled away exposing the adhesive tape, and the sides of the bag container near the mouth opening could then be pressed together to seal the bag container. Alternatively, an adhesive strip may be included along the outer surface for securing a first folded edge of the bag container to an opposite folded edge in a closed and folded configuration thereby preventing the bag from unfolding and opening inadvertently.

The closure means may also comprise a twist-tie member adhesively secured to an outer surface of the bag container that allows the upper portion of the bag container to be folded closed and secured using the flexible twist-tie member.

Other closure means exist in the art such as an envelope type overlapping adhesive configuration, a drawstring or handle ties, such as are commonly used on kitchen trash bags, and many others.

Enclosed within the container bag of the collection bag kit of the present invention is preferably a quantity of an absorbent material for absorbing the liquid contained within the bag. This absorbent material preferably contains a hydrophilic gellable material, usually a polymer, which is water activated and which gels very rapidly upon contact with a bodily fluid (such as emeses) and which by gelling completely absorbs and encapsulates substantially all of the fluid. Such polymers are commercially available and are commonly found in a variety of known products, including disposable-diapers and cleaning compositions. Typical examples include that described in U.S. Pat. No. 4,179,367. The gellable mixture may also include not only the gellable material but also a material such as a protease enzyme to attack and break down the bodily fluid to enhance the operation of the gellable material. Also included can be deodorants and fragrances. The gellable mixture may by in any convenient physical form which can be placed into the container bag; granular, powdered, foamed, matted, woven and fibrous forms are all suitable.

In the present invention, a cleansing towelette, or wipe, and a breath freshener is removably attached to an outside surface of the disposable bag container. More than one towelette or breath freshener may be included. The towelette or towelettes and breath freshener or breath fresheners may be attached to the same or different outside surfaces of the bag container.

The cleansing towelette is preferably pre-moistened with a wet cleanser, such as water; more preferably with a sanitizing or antimicrobial solution or gel.

The cleansing towelette, or wipe, may be made of standard construction and of standard material, including paper, cloth, and non-woven fabric comprised of synthetic fibers or natural fibers, and foamed material. The towelette may be a single ply or multi-ply sheet, or sponge like. Preferably for environmental reasons, the towelette or wipe (and its associated packet, if any) is made of biodegradable or compostable material. If the towelette is pre-moistened, it should be made of material that is absorbent of the cleanser, solution or gel employed to pre-moisten it.

The cleansing towelette or wipe may be any type that is generally available. It is preferred that the towelette or wipe be individual sealed in its own packet container. The material for the packet, if the cleansing wipe is pre-moistened, should be moisture proof, and preferably odor impermeable. The same or different materials as used to make the container bag may be used, if suitable.

Any sealing means of any suitable type may be employed to seal the cleansing towelette in its packet. The seal can be by means of heat or pressure sealing, or adhesive once the cleansing wipe inside its packet. Preferably, the packet is readily openable by tearing or by providing a ready open seal.

The towelette, or wipe, may be unscented or scented with any desired scent such as lemon, lavender, citrus, coconut, cherry, strawberry, and other pleasant natural scents.

The breath freshener may be of any type that is generally available. It may be in the form of an edible film, orally dissolving strip, chewing gum, tablet, lozenge, liquid, hard candy, or a gel. For reasons of ease of packaging, an orally dissolving strip is preferred.

Also preferred is that the breath freshener have a pleasant taste and smell such as peppermint, spearmint, cinnamon, cherry, strawberry, vanilla, piña-colada, or lemon.

It is preferred that the breath freshener be in its own package. Any packaging typical and suited to the form of the breath freshener may be employed, included a packet the same or similar to the packet container employed for the cleansing wipe. As with the container bag and cleansing towelette material, it is preferred that the breath freshener's packaging is made of biodegradable or compostable material.

The preferred means of removably attaching the cleansing towelette or wipe and the breath freshener to an outside surface of the bag container is to provide a peel-away cover layer over the items which are located on the outside surface of the bag container. The cover layer may be formed by placing the cleansing towelette and breath freshener against an outside surface of the bag container material, laying a sheet of the bag material or another material on top of the cleansing towelette and breath freshener, and then heat or pressure sealing the bag materials together. It is preferable that the bag container material is of an opaque material and the cover layer is of a transparent material.

The cover ply may consist of, but is not limited to, a polypropylene or a polyethylene film type material, or a similar material. Preferably the material is biodegradable or compostable.

A perforation may be provided for tear-away access to the cleansing wipe and breath freshener.

The provision of a cover layer may also be accomplished by using an adhesive to adhere the cover layer of bag material sheet to the outside surface of the bag container. The cleansing towelette and breath freshener would be placed and located on the outside of the bag container; a clear cover layer would then be applied on top of the items with the clear cover layer having an adhesive along the border of the cover. The cover would be adhered in a manner that would allow a peel-away of the cover ply to access the wipe and freshener items.

The cleansing wipe and breath freshener also may be removably attached to an outside surface of the bag container by means of heat or pressure sealing the outer edges of the packet, or packaging of the cleansing wipe and breath freshener to the bag container, or by means of the packet, or packaging having an adhesive on the outside thereof to adhere them to an outside surface of the bag container.

The cleansing wipe and breath freshener may also be attached to the collection bag container by means of a package containing both items. The package containing both items may be attached or fixed to the bag container in the same manner as where the cleansing towelette and breath freshener are separately attached. Alternatively, the package containing both items may be attached or fixed to the bag container by a connecting strap type means or a rubber band attachment that allows the package to be removed with the removal of the connecting strap or rubber band.

Other items may be included in the bodily fluid collection bag kit. To protect an included item from possible contact with the bodily fluid being collected and possible misplacement, and to have it readily accessible, it is preferred that the item (or items) is removably attached to an outside surface of the bag container. If the bag container has more than one outside surface, the additional item may be removably attached to the same outside surface to which the towelette or breath freshener is attached or a different outside surface. Attachment of any additional item may be by any suitable means, including those means suitable for removably attaching a towelette and breath freshener to the outside surface of the bag container. As with the bag container, breath freshener and cleansing towelette, it is preferred that all included items are made of biodegradable or compostable material.

Having thus described and exemplified the invention with a certain degree of particularity, it should be appreciated that the following claims are not to be so limited but are to be afforded a scope commensurate with the wording of each element of the claim and equivalents thereof.

What is claimed is:

1. A bodily fluid collection bag kit comprising:
a) a disposable, flexible bag container having a mouth opening,
b) a cleansing towelette removably attached to or contained on an outside surface of the bag container, and
c) a breath freshener removably attached to or contained on an outside surface of the bag container.

2. The bodily fluid collection bag kit of claim 1, further comprising a cover layer overlaying and containing the cleansing towelette or the breath freshener on an outside surface of the bag container.

3. The bodily fluid collection bag kit of claim 2, wherein the cover layer is heat or pressure sealed to the outside surface of the bag container.

4. The bodily fluid collection bag kit of claim 2, wherein the cover layer is adhesively adhered to the outside surface of the bag container.

5. The bodily fluid collection bag kit of claim 1, wherein the cleansing towelette or the breath freshener is removably attached to an outside surface of the bag container by an adhesive.

6. The bodily fluid collection bag kit of claim 1, further comprising an adsorbent material inside the bag container; wherein when a bodily fluid enters the bag container through the mouth opening, the absorbent material absorbs at least some of the bodily fluid.

7. The bodily fluid collection bag kit of claim 6, wherein the absorbent material comprises a gelling material, wherein when a bodily fluid enters the bag container through the mouth opening, the gelling material turns into a gel.

8. The bodily fluid collection bag kit of claim 7, wherein the gelling material is a cellulose material or a hydrophilic material.

9. The bodily fluid collection bag kit of claim 7, wherein the gelling material is a hydrophilic polymer.

10. The bodily fluid collection bag kit of claim 2, further comprising an adsorbent material inside the bag container; wherein when a bodily fluid enters the bag container through the mouth opening, the absorbent material absorbs at least some of the bodily fluid.

11. The bodily fluid collection bag kit of claim 1, wherein the bag container comprises a bottom edge opposite the mouth opening, and one or more side walls extending from the mouth opening to the bottom edge; and wherein the cleansing towelette and the breath freshener are removably attached to an outside surface of the one or more side walls.

12. The bodily fluid collection bag kit of claim 1, wherein the bag container comprises a bottom wall opposite the mouth opening, and one or more side walls extending from the mouth opening to the bottom wall; and wherein the cleansing towelette and the breath freshener are removably attached to an outside surface of the bottom wall.

13. A bodily fluid collection bag kit comprising:
1) a disposable, flexible bag container having an interior with
a) a mouth opening,
b) a bottom wall opposite the mouth opening,
c) one or more side walls extending from the mouth opening to the bottom wall and at least partially defining the interior of the bag container; the one or more side walls each having one or more inward folding pleats overlying the bottom wall;
2) a breath freshener removably attached to an outside surface of the bottom wall, and
3) a cleansing towelette removably attached to the outside surface of the bottom wall.

14. The bodily fluid collection bag kit of claim 13, further comprising a cover layer overlaying and containing the cleansing towelette or the breath freshener on an outside surface of the bottom wall of the bag container.

15. The bodily fluid collection bag kit of claim 13, further comprising an adsorbent material in the interior of the bag container; wherein when a bodily fluid enters the bag container through the mouth opening, the absorbent material absorbs at least some of the bodily fluid.

16. The bodily fluid collection bag kit of claim 14, further comprising an adsorbent material inside the bag container; wherein when a bodily fluid enters the bag container through the mouth opening, the absorbent material absorbs at least some of the bodily fluid.

17. A bodily fluid collection bag kit comprising:
1) a disposable, flexible bag container having an interior and comprising:
a) a mouth opening,
b) a bottom wall opposite the mouth opening,
c) a first side wall and a second opposing side wall each extending from the mouth opening to the bottom wall and at least partially defining the interior of the bag container; each of the first and second walls having a plurality of lateral, inwardly folding accordion pleats; the pleats being outwardly unfoldable to thereby elongate the first and second side walls,
the bag container having a collapsible state wherein the pleats of the first and second side wall overly the bottom wall, and the bag container having an expandable state wherein the pleats of the first and second side walls are at least partially unfolded, thereby elongating the first and second side walls and increasing the interior of the bag container;
2) a breath freshener removably attached to an outside surface of the bottom wall of the bag container, and
3) a cleansing towelette removably attached to the outside surface of the bottom wall of the bag container.

18. The bodily fluid collection bag kit of claim 17, further comprising a cover layer overlaying and containing the cleansing towelette or the breath freshener on an outside surface of the bottom wall of the bag container.

19. The bodily fluid collection bag kit of claim 17, further comprising an adsorbent material in the interior of the bag container; wherein when a bodily fluid enters the bag container through the mouth opening, the absorbent material absorbs at least some of the bodily fluid.

20. The bodily fluid collection bag kit of claim 18, further comprising an adsorbent material inside the bag container; wherein when a bodily fluid enters the bag container through the mouth opening, the absorbent material absorbs at least some of the bodily fluid.
